(19) United States
(12) Patent Application Publication
(10) Pub. No.: US 2025/0152507 A1
(43) Pub. Date: May 15, 2025

(54) SUBLINGUAL COMPOSITIONS FOR ERECTILE DYSFUNCTION

(71) Applicant: Woody's Medical LLC, Scottsdale, AZ (US)

(72) Inventors: Austin Lucht, Scottsdale, AZ (US); Suzanne Bentz, Scottsdale, AZ (US)

(73) Assignee: Woody's Medical LLC, Scottsdale, AZ (US)

(21) Appl. No.: 18/505,212

(22) Filed: Nov. 9, 2023

(51) Int. Cl. A61K 9/00; A61K 31/401; A61K 31/4985; A61K 31/53; A61K 47/02; A61K 47/14; A61K 47/32

(52) U.S. Cl. CPC A61K 9/006; A61K 31/401; A61K 31/4985; A61K 31/53; A61K 47/02; A61K 47/14; A61K 47/32; A61K 2121/00

(57) ABSTRACT

Sublingual compositions for the treatment of erectile dysfunction (ED) comprise unique combinations of phosphodiesterase-type 5 (PDE5) inhibitors and D1/D2 dopamine receptor agonists. In preferred embodiments, a combination of four drug actives exhibits a full range of effectiveness for ED in which there is a short and long acting PDE5 inhibitor in combination with both a dopamine agonist and a CNS hormone to provide both a physiological response and a psychological/CNS action. A treatment for ED comprises administration of sublingual compositions in the physical form of sublingual tablets.

### FIELD

This disclosure generally relates to pharmaceutical compositions usable to treat erectile dysfunction, and more specifically, to sublingually administered tableted compositions comprising one or a combination of drug actives for treatment of erectile dysfunction.

### BACKGROUND

Erectile dysfunction (ED) is a physiological condition in which a male individual is unable to achieve or suitably sustain an erection sufficient for sexual intercourse. ED can be caused by a number of medical issues, most notably age, illness, current medications, physical injury, and insufficient blood flow. Many men experiencing ED have underlying comorbidities that are risk factors for erectile dysfunction, including for example diabetes, hypertension, cardiovascular disease, depression, prostatic hypertrophy, smoking, drug treatment, sedentary lifestyle, and drug and alcohol abuse.

Several phosphodiesterase-type 5 (PDE5) inhibitors are available to treat ED. The most notable being sildenafil (Viagra®), tadalafil (Cialis®), vardenafil (Levitra®), and avanafil (Stendra®). Although at least these four medications are available as oral tablets, the dosage levels for each need to be high to compensate for poor intestinal absorption, which adds to the cost per tablet and the likelihood for gastrointestinal problems. Further, oral administration is an inherently slow route to deliver drug actives systemically, which is simply unacceptable for ED medications.

Apomorphine is a D1/D2 dopamine receptor agonist and is the first centrally acting treatment for ED. However, it has been reported to have only a small effect in men with mild ED, and it is unlikely to be effective in men who do not respond to sildenafil. See C. McMahon, et al., “Treating erectile dysfunction when PDE5 inhibitors fail,” BMJ, 2006 Mar. 11; 332(7541): 589-592.

In view of these and other shortcomings, there remains an unmet need for faster acting and more cost effective ED medications that are easy to administer.

### SUMMARY

It has now been surprisingly discovered that certain PDE5 inhibitors and dopamine receptor agonists, compounded singly or in certain combinations, can be administered to individuals sublingually for rapid ED treatment. Sublingually delivered vardenafil, tadalafil, apomorphine, and oxytocin, singly or in various combinations, are found to work within the central nervous system (CNS) either as hormone or dopamine agonists to provide male erection faster than what is currently achievable from existing oral ED medications. The novel formulation strategy disclosed herein reduces the required levels of drug actives per dose, thus reducing cost per tablet and mitigating gastrointestinal issues such as indigestion and diarrhea commonly experienced from orally administered ED medications.

This disclosure describes sublingual compositions usable to treat ED. In various embodiments, these compositions are compounded in solid form, such as in the form of free-flowing powders that are amenable to tableting in a tablet press.

In various embodiments, sublingual compositions for ED in accordance with the present disclosure comprise at least one of vardenafil, tadalafil, apomorphine, and oxytocin, and a carrier configured for a powdered composition amendable to compression into individual sublingual dosage tablets.

In various examples, a sublingual composition for ED comprises oxytocin as the sole drug active formulated in single dose tablets configured for sublingual administration.

In nonlimiting examples, a sublingual tablet for ED weighs about 100 mg and delivers from about 0.042 mg to about 0.126 mg oxytocin, preferably about 0.084 mg oxytocin (50 IU). The remainder of the sublingual tablet for ED comprises a carrier further comprising at least one excipient.

In various examples, a sublingual composition for ED comprises a combination of tadalafil and oxytocin formulated in single dose tablets configured for sublingual administration.

In nonlimiting examples, a sublingual tablet for ED weighs about 275 mg and delivers from about 2.5 mg to about 20 mg tadalafil, preferably about 12.5 mg tadalafil, and from about 0.0168 mg to about 0.084 mg oxytocin, preferably about 0.042 mg oxytocin, in combination. The remainder of the sublingual tablet for ED comprises a carrier further comprising at least one excipient.

In various examples, a sublingual composition for ED comprises a combination of apomorphine, vardenafil and oxytocin formulated in single dose tablets configured for sublingual administration.

In nonlimiting examples, a sublingual tablet for ED weighs about 275 mg and delivers about 0.042 mg oxytocin, 10 mg vardenafil, and 2 mg apomorphine, in combination. The remainder of the sublingual tablet for ED comprises a carrier further comprising at least one excipient.

In various examples, a sublingual composition for ED comprises a combination of tadalafil, apomorphine, vardenafil and oxytocin formulated in single dose tablets configured for sublingual administration. This unique combination of four drug actives exhibits a full range of effectiveness in which there is a short and long acting PDE5 inhibitor in combination with both a dopamine agonist and CNS hormone to provide both a physiological response with a mental/CNS action. This unique combination has the capability to treat not only the physiological aspect of ED but the mental/cognitive aspects of ED, as it has two PDE5 inhibitors and cognitive medications in combination.

In nonlimiting examples, a sublingual tablet for ED weighs about 275 mg and delivers about 10 mg tadalafil, about 0.042 mg oxytocin, about 10 mg vardenafil, and about 2 mg apomorphine, in combination. The remainder of the sublingual tablet for ED comprises a carrier further comprising at least one excipient.

In various embodiments, a carrier for a sublingual tablet for ED comprises a filler, a binder, a lubricant, a disintegrant, a glidant, or mixtures thereof. These excipients in the carrier are optimized for rapid disintegration, dissolution and transmucosal absorption under the tongue.

In various embodiments, the carrier comprises a mixture of untreated fumed silica, sodium stearyl fumarate, microcrystalline cellulose, colloidal silicon dioxide, mannitol, fructose, and crosslinked polyvinylpyrrolidone (“crospovidone”).

In various embodiments, a carrier for a sublingual tablet for ED comprises from about 0.1 wt. % to about 5 wt. % of sodium stearyl fumarate.

In various embodiments, a carrier for a sublingual tablet for ED comprises from about 0.5 wt. % to about 1 wt. % untreated fumed silica.

In various embodiments, a carrier for a sublingual tablet for ED comprises from about 0.1 wt. % to about 5 wt. % crospovidone.

In various embodiments, a sublingual tablet for sublingual treatment of ED comprises from about 0.10 wt. % to about 10.0 wt. % in total of at least one of tadalafil, apomorphine, vardenafil, and oxytocin, with the remainder of the sublingual tablet comprising a carrier further comprising untreated fumed silica, sodium stearyl fumarate, microcrystalline cellulose, colloidal silicon dioxide, mannitol, fructose and crospovidone, wherein the weight percentages are based on the total weight of the tablet.

In various embodiments, each sublingual tablet comprises from about 2.0 wt. % to about 5.0 wt. % tadalafil, from about 2.0 wt. % to about 5.0 wt. % vardenafil, from about 0.01 wt. % to about 0.10 wt. % oxytocin, and/or from about 0.5 to about 1.0 wt. % apomorphine, with the remainder of each sublingual tablet being a carrier.

In nonlimiting examples, each tablet weighs approximately 275 mg.

In various embodiments, a method for treating ED in an individual in need thereof comprises sublingually administering to the individual a therapeutically effective amount of a sublingual composition for ED comprising from about 2.0 wt. % to about 5.0 wt. % tadalafil, from about 2.0 wt. % to about 5.0 wt. % vardenafil, from about 0.01 wt. % to about 0.10 wt. % oxytocin, and/or from about 0.5 to about 1.0 wt. % apomorphine; and remainder, a carrier, wherein the weight percentages are based on the total weight of the sublingual composition for ED.

In various embodiments, a therapeutically effective amount is an amount sufficient of the sublingual composition for ED to deliver a total of from about 2.5 mg to about 20 mg tadalafil, from about 2.5 mg to about 20 mg vardenafil, from about 0.0168 mg (10 IU) to about 0.084 mg (50 IU) oxytocin, and/or from about 1 mg to about 3 mg apomorphine.

In various embodiments, a sublingual composition for ED is in the physical form of individual sublingual tablets weighing approximately 100 mg or 275 mg each, and wherein the therapeutically effective amount of the sublingual composition for ED comprises from about one-half (½) of one sublingual tablet to about five (5) sublingual tablets.

In various embodiments, a sublingual composition for erectile dysfunction (ED) comprises from about 0.01 wt. % to about 0.10 wt. % of oxytocin actives, optionally in combination with at least one other active selected from the group consisting of vardenafil, tadalafil, apomorphine, and mixtures thereof; and a carrier; wherein the weight percentages are based on the total weight of the sublingual composition for erectile dysfunction (ED), and wherein the sublingual composition is in the physical form of a loose powder amendable to tableting by compression in a tablet press.

In various embodiments, the sublingual composition for erectile dysfunction (ED) comprises from about 0.05 wt. % to about 0.10 wt. % oxytocin in the absence of said optional actives in combination, based on the total weight of the sublingual composition for erectile dysfunction (ED).

In various embodiments, the sublingual composition for erectile dysfunction (ED) comprises from about 2.0 wt. % to about 5.0 wt. % tadalafil and from about 0.01 wt. % to about 0.02 wt. % oxytocin in combination, based on the total weight of the sublingual composition for erectile dysfunction (ED).

In various embodiments, the sublingual composition for erectile dysfunction (ED) comprises from about 2.0 wt. % to about 5.0 wt. % vardenafil, from about 0.01 wt. % to about 0.02 wt. % oxytocin, and from about 0.5 to about 1.0 wt. % apomorphine in combination, based on the total weight of the sublingual composition for erectile dysfunction (ED).

In various embodiments, the sublingual composition for erectile dysfunction (ED) comprises from about 2.0 wt. % to about 5.0 wt. % tadalafil, from about 2.0 wt. % to about 5.0 wt. % vardenafil, from about 0.01 wt. % to about 0.02 wt. % oxytocin, and from about 0.5 to about 1.0 wt. % apomorphine in combination, based on the total weight of the sublingual composition for erectile dysfunction (ED).

In various embodiments, the carrier comprises a filler, a binder, a lubricant, a disintegrant, a glidant, or mixtures thereof.

In various embodiments, the carrier comprises a mixture of untreated fumed silica, sodium stearyl fumarate, microcrystalline cellulose, colloidal silicon dioxide, mannitol, fructose, and crospovidone.

In various embodiments, the carrier comprises from about 0.1 wt. % to about 5 wt. % of sodium stearyl fumarate, based on the total weight of the sublingual composition for erectile dysfunction (ED).

In various embodiments, the carrier comprises from about 0.5 wt. % to about 1 wt. % untreated fumed silica, based on the total weight of the sublingual composition for erectile dysfunction (ED).

In various embodiments, the carrier comprises from about 0.1 wt. % to about 5 wt. % crospovidone, based on the total weight of the sublingual composition for erectile dysfunction (ED).

In various embodiments, a sublingual tablet for transmucosal administration comprises from about 2.0 wt. % to about 5.0 wt. % tadalafil; from about 2.0 wt. % to about 5.0 wt. % vardenafil; from about 0.01 wt. % to about 0.02 wt. % oxytocin; from about 0.5 to about 1.0 wt. % apomorphine; and at least about 85 wt. % of a carrier; wherein the weight percentages are based on the total weight of the sublingual tablet.

In various embodiments, the carrier comprises a mixture of untreated fumed silica, sodium stearyl fumarate, microcrystalline cellulose, colloidal silicon dioxide, mannitol, fructose and crospovidone.

In various embodiments, each tablet weighs approximately 275 mg and wherein each tablet delivers about 0.042 mg oxytocin, 10 mg vardenafil, 10 mg tadalafil, and 2 mg apomorphine.

In various embodiments, the carrier further comprises at least one of a sweetener and flavoring agent.

In various embodiments, a method for treating erectile dysfunction (ED) in an individual in need thereof comprises sublingually administering to the individual a therapeutically effective amount of a sublingual composition comprising from about 2.0 wt. % to about 5.0 wt. % tadalafil; from about 2.0 wt. % to about 5.0 wt. % vardenafil; from about 0.01 wt. % to about 0.02 wt. % oxytocin; from about 0.5 to about 1.0 wt. % apomorphine; and remainder, a carrier; wherein the weight percentages are based on the total weight of the sublingual composition.

In various embodiments, the therapeutically effective amount is an amount sufficient of the sublingual composition to deliver 2.5 mg to 20 mg vardenafil, 2.5 mg to 20 mg tadalafil, 1 mg to 3 mg apomorphine, and 0.0168 mg to 0.084 mg oxytocin.

In various embodiments, the sublingual composition is in the physical form of individual sublingual tablets weighing approximately 275 mg each, and wherein the therapeutically effective amount of the sublingual composition comprises from about one (1) to about five (5) sublingual tablets, taken as needed.

In various embodiments, the individual in need thereof is clinically evaluated to have erectile dysfunction (ED).

In various embodiments, a sublingual tablet consists essentially of 2.5 mg oxytocin; 10 mg vardenafil; 2 mg apomorphine; and 260.5 mg of a mixture of fumed untreated silica, sodium stearyl fumarate, microcrystalline cellulose, colloidal silicon dioxide, mannitol, fructose, crospovidone, optional sweetener, and optional flavoring agent.

In various embodiments, a sublingual tablet consists essentially of 2.5 mg oxytocin; 12.5 mg tadalafil; and 260.0 mg of a mixture of fumed untreated silica, sodium stearyl fumarate, microcrystalline cellulose, colloidal silicon dioxide, mannitol, fructose, crospovidone, optional sweetener, and optional flavoring agent.

### DETAILED DESCRIPTION

The detailed description of exemplary embodiments refers to the accompanying drawings, which show exemplary embodiments by way of illustration and best mode. While these exemplary embodiments are described in enough detail to enable those skilled in the art to practice the invention, other embodiments may be realized, and logical, chemical, and mechanical changes may be made without departing from the spirit and scope of the inventions. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

Sublingual compositions for the treatment of erectile dysfunction (ED) are described, along with dosage sizes and regimens, and methods of treating ED using the present compositions. In various embodiments, sublingual ED compositions in powdered form are optimized for compression into single dose sublingual tablets configured for rapid disintegration and transmucosal permeation of drug actives across sublingual mucosa, allowing for a substantial reduction in drug active amounts necessary for a physiologically effect of rapid male erection and endurance thereof, and minimization of gastrointestinal side effects.

In preferred embodiments, a sublingual composition for ED is provided in a tablet dosage form for sublingual administration. In various examples, a sublingual composition for

ED is in the form of a loose or free-flowing powder that can be compressed in a tablet press into sublingual tablets weighing about 50 mg to about 400 mg and preferably about 100 mg to 275 mg each.

### DEFINITIONS

As used herein, the term “oxytocin” refers to the peptide precursor encoded by the human OXT gene, having a molecular weight of 1007.2 g/mol and a molecular formula C₄₃H₆₆N₁₂O₁₂S₂, and assigned CAS No. 50-56-6. This substance is amply described in PubChem, National Library of Medicine of the National Institutes of Health, under PubChem CID 439302. Also for use herein are various salts of oxytocin, such as the monoacetate salt, and prodrugs of oxytocin. Amounts of oxytocin are sometimes expressed in International Units (IU). For oxytocin, 1 IU is equivalent to 1.68 μg of the pure peptide. For purposes herein, a convenient ingredient to use in compounding sublingual tablets is “OXYTOCIN 10 UNIT/MG/MANNITOL TRITURATION,” which contains 16.8 μg oxytocin per milligram, or 0.0168 mg/1 mg or 1.68 wt. % oxytocin based on the total weight of this commercial ingredient. Thus, for example, a sublingual tablet for ED containing 2.5 mg of “OXYTOCIN UNIT/MG/MANNITOL TRITURATION” ingredient delivers 25 IU oxytocin, or 42 μg oxytocin (0.042 mg).

As used herein, the term “tadalafil” refers to the indole alkaloid having the structure (I):

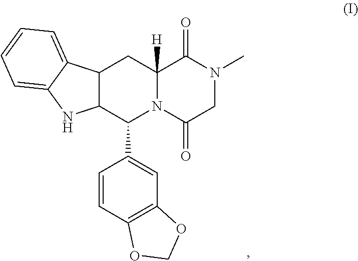 [SMILES: CN1CC(=O)N2[C@H](CC3c4ccccc4NC3[C@H]2c2ccc3c(c2)OCO3)C1=O]

having molecular formula C₂₂H₁₉N₃O₄ and assigned CAS No. 171596-29-5. This compound is typically synthesized from (D)-tryptophan methyl ester and piperonal via a Pictet-Spengler reaction followed by sequential condensation reactions with chloroacetyl chloride and methylamine, respectively, to close the diketopiperazine ring. Tadalafil is a phosphodiesterase (PDE) inhibitor. Also included for use herein are various salts of tadalafil, such as the HCl salt, and prodrugs of tadalafil.

As used herein, the term “vardenafil” refers to the alkaloid having the structure (II):

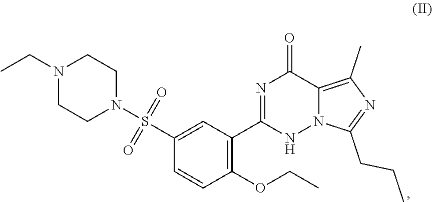 [SMILES: CCCc1nc(C)c2c(=O)nc(-c3cc(S(=O)(=O)N4CCN(CC)CC4)ccc3OCC)[nH]n12]

having molecular formula C₂₃H₃₂N₆O₄S and assigned CAS No. 224785-90-4. Also included for use herein are various salts of vardenafil, such as the HCl salt, and prodrugs of vardenafil.

As used herein, the term “apomorphine” refers to the dopamine agonist having the structure (III):

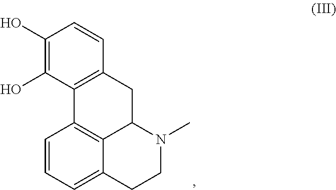 [SMILES: CN1CCc2cccc3c2C1Cc1ccc(O)c(O)c1-3]

having molecular formula C₁₇H₁₇NO₂ and assigned CAS No. 58-00-4. Also included for use herein are various salts of apomorphine, such as the HCl salt, and prodrugs of apomorphine.

As used herein, the term “excipient” takes on its ordinary meaning in pharmaceutical compounding, namely the ingredients in a pharmaceutical composition other than drug actives (i.e., the inactive ingredients said to make up the composition of a “carrier”). In pharmaceutical tablets, excipients include, for example, diluents (sometimes called inert fillers), binders, disintegrants, lubricants, glidants, acidic agents, alkaline agents, pH buffering agents, colorings, flavorings, sweeteners, preservatives, surfactants, drug release-modifying agents, and coating materials. For sublingual tablets, drug release modifiers (such as to slow the release of a drug active in the gut) and tablet coatings (such as to ease swallowing) are not important, whereas disintegrants are important, seeing that a sublingual tablet generally needs to disintegrate under the tongue in a relatively short period of time and the drug actives need to absorb quickly. Some excipients used in tablet compositions may provide a benefit to the tableting process and overall manufacturing operation (e.g., binders and glidants). Certain excipients may promote flow of a powder into a tablet press, and/or optimize the compression process, tablet hardness, and so forth. It should be appreciated that certain chemical entities may provide multiple benefits, such as being both inert filler and a binding agent, and thus certain excipients for pharmaceutical tablets may fit into more than one functional group (filler, binder, disintegrant, etc.). For simplicity in describing the sublingual tablets herein, excipients may be said to be present in total in a “quantity sufficient” or “q. s.” to bring a composition up to a total of 100 wt. %. The totality of the excipients may also be referred to as the “carrier” in a tablet composition. In various embodiments, a carrier herein comprises at least one excipient.

As used herein, the name “PROSOLV® ODT G2” refers to a commercially available product from JRS Pharma, Rosenberg, Germany, used in orally disintegrating tablet formulation, development and manufacture. This product provides a mixture of microcrystalline cellulose, colloidal silicon dioxide, mannitol, fructose, and crospovidone (poly-N-vinyl-2-pyrrolidone), wherein the combined amount of microcrystalline cellulose, colloidal silicon dioxide, mannitol and fructose is about 95 wt. % to about 98 wt. % of the total PROSOLV® ODT G2 mixture, and wherein the amount of crosprovidone is about 2 wt. % to about 5 wt. % of the total PROSOLV® ODT G2 mixture. Use of PROSOLV® ODT G2 streamlines tablet manufacturing since it includes each of these functional materials in a single mixture, thus eliminating the need to add each functional component separately. In certain embodiments, individual functional ingredients can be compounded separately into a composition rather than using the PROSOLV® ODT G2 mixture.

As used herein, the term “composition” takes on the ordinary meaning in formulation chemistry as a combination of ingredients. Simply written, a pharmaceutical composition may comprise one or more drug actives and a carrier, with the carrier comprising a suitable blend of excipients such that the final composition has the desired properties. In various embodiments, a composition is designed to adopt a particular physical form, or at least be amenable to physical manipulation into a desired physical form, which may be the dosage form for a particular treatment regimen. For example, a composition may be a free-flowing powder that by virtue of the selection of excipients, is amenable to tableting on a tablet press. Typically, a solid composition is made homogeneous by mixing or blending, such as in a ribbon blender, although not all powder compositions are white and/or homogeneous as to particle size distribution. In various embodiments, a composition herein comprises a powdered mixture amenable to compression on a tablet press into small tablets suitable for sublingual administration, wherein the administration route is characterized by rapid disintegration of the tablet under the tongue.

Ingredients for a composition herein are generally shown “as added,” meaning there is a possibility for one or more chemical reactions between ingredients once the ingredients are mixed together, such as into a common carrier. One skilled in the art of formulation chemistry can recognize whether ingredients might react in a mixture. These reactions can include neutralization (e.g., between acid and alkali ingredients), mixed micelle formation (mixed surfactants in liquid systems) or other encapsulation phenomena, hydrolysis, and so forth. In various embodiments, ingredients in a composition are listed in “weight percent,” (i.e., “wt. %”), based on the total weight of the composition. For example, 100 milligrams of a composition comprising 40 mg A and 60 mg B may be recited as “40 wt. % A and 60 wt. % B, based on the total weight of the composition,” which necessarily totals to “100 wt. %.” The actual weight amounts, (e.g., milligrams or grams) generally refers to amounts added for a particular batch size (e.g., a batch size of powder usable to make 100, 1000, or 10,000, or more, tablets).

As used herein, the term “dosage form” takes on its ordinary meaning in the pharmaceutical arts as the physical form of a composition designed for a particular administration route. For sublingual tablets herein, the dosage form is a tablet amenable to sublingual administration and disintegration. In various nonlimiting examples, an individual tablet herein may weigh from about 150 to about 350 mg, preferably about 275 mg, and each tablet may provide (a) about 0.084 mg oxytocin; or (b) about 12.5 mg tadalafil and about 0.042 mg oxytocin; or (c) about 2 mg apomorphine, about 10 mg vardenafil, and about 0.042 mg oxytocin; or (d) about 10 mg tadalafil, about 2 mg apomorphine, about 10 mg vardenafil, and about 0.042 mg oxytocin, with the remainder of each tablet (a), (b), (c) or (d) being a carrier that comprises at least one excipient.

As used herein, the terms “subject,” or “individual,” or the phrases “a subject in need thereof,” or “an individual in need thereof,” refers to any human or non-human animal requiring or desirous of a pharmacological change. For example, a subject in need thereof may be a human patient clinically diagnosed with ED, a circulatory issue, or a health issue relating to or believed to cause diminished sexual performance, be it a physical or psychological health issue or a combination of physical and mental factors. In various embodiments, the subject in need thereof is a person desirous of improved sexual performance, libido, or confidence. Most importantly, a subject in need thereof can be any human in relatively good health, but desirous of improved sexual performance.

As used herein, the term “treatment” of a subject (e.g., a human in need thereof) is any type of intervention used in an attempt to alter the natural course of the subject. Treatment includes, for example, administration of a sublingual composition for ED and that administration may be several minutes before sexual intercourse.

As used herein, the term “therapeutically effective amount” refers to a minimum dosage of a composition that provides a desired or an expected outcome, such as achievement of an erection, the strength or perceived “hardness” of an erection, and/or a particular length of time an erection is maintained, or some subjective improvement in sexual performance as perceived by the individual, such as compared subjectively by the individual to a previous sexual performance absent any treatment. A therapeutically effect amount of a sublingual tablet for ED herein may be one, or two, or several 275 mg tablets, administered sublingually, just prior to a sexual encounter.

As used herein, the term “modulate” includes to “increase” or “decrease” one or more quantifiable parameters, optionally by a defined and/or statistically significant amount. By “increase” or “increasing,” “enhance” or “enhancing,” or “stimulate” or “stimulating,” refers generally to the ability of one or more sublingual compositions for ED to produce or cause a greater physiological response (i.e., downstream effects) in a cell or in a subject relative to the response caused by either no ED composition or a control compound. Relevant physiological or cellular responses (in vivo or in vitro) upon administration of a sublingual tableted ED composition will be apparent to persons skilled in the art. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times), including all integers and decimal points in between and above 1 (e.g., 1.5, 1.6, 1.7. 1.8), the amount produced by no ED treatment composition (the absence of a pharmaceutical active) or a control compound. Relevant physiological or cellular responses (in vivo or in vitro) will be apparent to persons skilled in the art.

As used herein, the term “approximately” in reference to amounts refers to plus or minus 5% of the value given, such as wt. %. The term “about” in reference to amounts such as weight refers to plus or minus 10% of the value given, such as a value expressed in wt. %.

### GENERAL EMBODIMENTS

### Drug Actives

In various embodiments, sublingual compositions for ED in accordance with the present disclosure comprise at least one pharmaceutical active selected from the group consisting of vardenafil, tadalafil, apomorphine, oxytocin, and mixtures thereof, and a carrier configured for a powdered composition amendable to compression into individual sublingual dosage tablets.

In various examples, a sublingual composition for ED comprises oxytocin as the sole drug active formulated in single dose tablets configured for sublingual administration.

In various examples, a sublingual composition for ED comprises a combination of tadalafil and oxytocin formulated in single dose tablets configured for sublingual administration.

In various examples, a sublingual composition for ED comprises a combination of apomorphine, vardenafil and oxytocin formulated in single dose tablets configured for sublingual administration.

In various examples, a sublingual composition for ED comprises a combination of tadalafil, apomorphine, vardenafil and oxytocin formulated in single dose tablets configured for sublingual administration. This unique combination of four drug actives exhibits a full range of effectiveness in which there is a short and long acting PDE5 inhibitor in combination with both a dopamine agonist and CNS hormone to provide both a physiological response with a mental/CNS action.

In various embodiments, each sublingual tablet, or a powdered composition for the treatment of ED that is amenable to tableting into sublingual tablets, comprises from about 2.0 wt. % to about 5.0 wt. % tadalafil, from about 2.0 wt. % to about 5.0 wt. % vardenafil, from about 0.01 wt. % to about 0.02 wt. % oxytocin, and from about 0.5 to about 1.0 wt. % apomorphine.

In various embodiments, each sublingual tablet, or a powdered composition for the treatment of ED that is amenable to tableting into sublingual tablets, comprises from about 2.0 wt. % to about 5.0 wt. % vardenafil, from about 0.01 wt. % to about 0.02 wt. % oxytocin, and from about 0.5 to about 1.0 wt. % apomorphine.

In various embodiments, each sublingual tablet, or a powdered composition for the treatment of ED that is amenable to tableting into sublingual tablets, comprises from about 2.0 wt. % to about 5.0 wt. % tadalafil and from about 0.01 wt. % to about 0.02 wt. % oxytocin.

In various embodiments, each sublingual tablet, or a powdered composition for the treatment of ED that is amenable to tableting into sublingual tablets, comprises from about 0.05 wt. % to about 0.10 wt. % oxytocin.

### Carrier

In various embodiments, a sublingual composition for the treatment of ED comprises at least 80 wt. % of a carrier, based on the total weight of the sublingual composition for the treatment of ED. In certain examples, a sublingual composition for the treatment of ED comprises >80.0 wt. %, >81.0 wt. %, >82.0 wt. %, >83.0 wt. %, >84.0 wt. %, >85.0 wt. %, >86.0 wt. %, >87.0 wt. %, >88.0 wt. %, >89.0 wt. %, or >90.0 wt. %, of a carrier, based on the total weight of the sublingual composition for the treatment of ED. A carrier for a sublingual composition for the treatment of ED is a solid substance, or more preferably, a mixture of solid substances, preferably in the form of a loose powder that is amenable to compressing into tablets.

In certain examples, a carrier for a sublingual composition for the treatment of ED comprises any combination of filler, binder, disintegrant, lubricant, glidant, surfactant, acidic agent, alkaline agent, pH buffering agent, preservative, drug release modifier, coating material, coloring, flavoring, and sweetener, totaling at least about 80 wt. % of the total weight of the sublingual composition for the treatment of ED.

In certain embodiments, a carrier for a sublingual composition for the treatment of ED comprises a filler and a disintegrant. In most cases, the filler represents the majority of the weight of a carrier.

In certain examples, a carrier for a sublingual composition for the treatment of ED comprises a filler, a lubricant, a glidant, and a disintegrant, where the filler represents the majority of the weight of a carrier.

### Filler (Diluent)

In various embodiments, a carrier for a sublingual composition for the treatment of ED comprises a filler. In certain examples, the filler will comprise the majority of the carrier by wt. %. Fillers include, for example, colloidal anhydrous silica (e.g., Aerosil® 200 or 300 Pharma, or Aeroperl® 300 Pharma), magnesium aluminometasilicate (Neusilin® US2), calcium silicate (Florite®), microcrystalline cellulose powder (e.g., Avicel® PH101 or PH102), silicified microcrystalline cellulose (PROSOLV® SMCC), various starches, and various sugars. Commonly used fillers in pharmaceutical powders and tablets include microcrystalline cellulose (MCC) and/or silicified microcrystalline cellulose (SMCC), and/or a simple sugar, such as mannitol, fructose, or lactose, or combinations of a cellulose and a simple sugar, such as a combination of microcrystalline cellulose and fructose (with either one as the majority wt. %). In various embodiments, a combination of MCC and colloidal silicon dioxide (e.g., 98:2) can substitute for SMCC, although there is some evidence the physical mixture results in some sticking of die punches (see, for example, A. Aljaberi, et al., “Functional performance of silicified microcrystalline cellulose versus microcrystalline cellulose: a case study,” Drug Dev. Ind. Pharma., 35 (9), 1066-1071, (2009)).

Other fillers that may find use in sublingual composition for the treatment of ED include, for example, cellulose acetate, sorbitol, sucrose, dextrin, dextrose, calcium phosphate dibasic, calcium carbonate, maltose, maltodextrin, kaolin, tribasic calcium phosphate, calcium sulfate, cellulose acetate butyrate (cellaburate), calcium lactate, cellulose acetate, erythritol, ethyl cellulose, ethyl acrylate/methyl methacrylate copolymer, isomalt, α-lactalbumin, lactitol, magnesium carbonate, magnesium oxide, methacrylic acid/ethyl acrylate copolymer, methacrylic acid/methyl methacrylate copolymer, polydextrose, sodium chloride, simethicone, hydrogenated pullulan, talc, amino methacrylate copolymer, trehalose, and xylitol.

In various embodiments, a carrier for a sublingual composition for the treatment of ED comprises a combination of microcrystalline cellulose, colloidal silicon dioxide, mannitol and fructose as a filler, at a level of from about 70 wt. % to about 90 wt. %, based on the total weight of the sublingual composition for the treatment of ED.

### Lubricant

In various embodiments, a carrier for a sublingual composition for the treatment of ED comprises a lubricant. Examples of a lubricant include, for example, magnesium stearate, calcium stearate, zinc stearate, sodium lauryl sulfate, sodium stearyl fumarate (SSF), magnesium lauryl sulfate, stearic acid, glyceryl behenate, behenoyl polyoxyl glycerides (e.g., Compritol® HD5), glyceryl dibehenate, lauric acid, glyceryl monostearate, glyceryl tristearate, myristic acid, palmitic acid, poloxamer, polyethylene glycol, polysorbate 20, polyoxyl-10-oleyl ether, polyoxyl-15-hydroxystearate, polysorbate 40, polyoxyl-20-cetostearyl ether, polyoxyl-40-stearate, polysorbate 60, polysorbate 80, potassium benzoate, sodium benzoate, sorbitan monolaurate, sorbitan monooleate, sodium stearate, sorbitan monopalmitate, sorbitan monostearate, zinc stearate, sorbitan sesquioleate, sorbitan trioleate, and talc.

In various embodiments, a carrier for a sublingual composition for the treatment of ED comprises sodium stearyl fumarate (SSF) as a tablet lubricant at a level of from about 0 wt. % to about 5 wt. %, based on the total weight of the sublingual composition for the treatment of ED. SSF is available, for example, from JRS Pharma under the trade name PRUV®, or from SPI Pharma under the brand name Lubripharm® SSF.

### Binder

In various embodiments, a carrier for a sublingual composition for the treatment of ED comprises a binder. Examples of a binder include, for example, polyvinylpyrrolidone (PVP, or povidone), crosslinked polyvinylpyrrolidone (crospovidone) vinylpyrrolidone-vinyl acetate copolymer (copovidone), carbomer, polyethylene glycol (PEG), starches and starch derivatives such as corn starch, pregelatinized starch, carboxymethylcellulose (carmellose), hydroxypropyl methylcellulose (hypromellose), cellulose ethers (e.g., Methocel™), hydroxyethyl cellulose, hydroxypropyl cellulose, hydroxyethyl methyl cellulose, calcium carboxymethylcellulose, calcium cellulose glycolate, ethyl cellulose, chitosan, dextrin, inulin, magnesium aluminum silicate, maltodextrin, methylcellulose, dextrates, sodium alginate, zein, gelatin, polymethacrylates, sorbitol, and acacia.

In various embodiments, a carrier for a sublingual composition for the treatment of ED comprises from none to up to about 20 wt. % binder.

### Disintegrant

In various embodiments, a carrier for a sublingual composition for the treatment of ED comprises a disintegrant. Examples of a disintegrant include, for example, crospovidone, croscarmellose sodium, sodium starch glycolate, crosslinked sodium carboxymethyl cellulose, low-substituted hydroxpropylcellulose, guar gum, chitosan hydrochloride, calcium alginate, sodium alginate, docusate sodium, magnesium aluminum silicate, methylcellulose, calcium carboxymethylcellulose, and calcium cellulose glycolate.

In various embodiments, a carrier for a sublingual composition for the treatment of ED comprises from about 0.1 wt. % to about 5 wt. % crospovidone, based on the total weight of the sublingual composition for the treatment of ED.

### Glidants

In various embodiments, a carrier for a sublingual composition for the treatment of ED comprises a glidant. Examples of a glidant include, for example, untreated fumed silica (e.g., CAB-O-SIL® M-5P or M-5DP), colloidal silicon dioxide, talc, tribasic calcium phosphate, calcium silicate, magnesium oxide, sodium stearate, magnesium silicate, magnesium trisilicate, and hydrophobic colloidal silica. In preferred embodiments, untreated fumed silica is included in a carrier for a sublingual composition for the treatment of ED as a glidant for improving tablet production. In instances where the tablet size is small (e.g., ≤100 mg), the additional lubrication from a glidant such as fumed silica might not be needed, and the excipient can be eliminated from the composition. Formulation 4 in TABLE 2, tableted at 100 mg/tablet, is an example.

In various embodiments, a carrier for a sublingual composition for the treatment of ED comprises untreated fumed silica as a glidant at a level of from about 0 wt. % to about 1 wt. %, based on the total weight of the sublingual composition for the treatment of ED. The untreated fumed silica acts as a free-flow agent to improve tablet production efficiencies, tablet uniformity, and tablet hardness.

In various embodiments, a carrier for a sublingual composition for the treatment of ED consists essentially of a mixture of untreated fumed silica, sodium stearyl fumarate, microcrystalline cellulose, colloidal silicon dioxide, mannitol, fructose and crospovidone, at a total level of at least about 80 wt. %, based on the total weight of the sublingual composition.

In various embodiments, a commercially available carrier for use in pharmaceutical tablets may be used as all or part of a carrier in a sublingual composition for the treatment of ED in accordance with the present disclosure. For example, PROSOLV® ODT G2 may be used as the carrier or as the majority of a carrier by weight for sublingual composition for the treatment of ED for tableting in accordance with the present disclosure. This commercial product, having a bulk density of about 0.45-0.65 g/mL, comprises a mixture of microcrystalline cellulose, colloidal silicon dioxide, mannitol, fructose, and crospovidone, and is available from JRS Pharma, Cedar Rapids, IA.

In various embodiments, a carrier for a sublingual composition for the treatment of ED comprises from about 80 wt. % to about 87 wt. % of PROSOLV®ODT G2. In various embodiments, a carrier for a sublingual composition for the treatment of ED comprises from about 80 wt. % to about 85 wt. % of PROSOLV®ODT G2 along with about 0.5 wt. % to 1 wt. % untreated fumed silica and about 0.1 wt. % to about 5 wt. % sodium stearyl fumarate. In various formulations, the weight percentage of PROSOLV®ODT G2 may be indicated simply as “quantity sufficient,” or “q.s.” In some examples, the amount of this carrier can be adjusted accordingly to accommodate changes in formulation, such as for example, presence or absence of a flavoring agent or sweetener, or higher or lower pharmaceutical drug actives levels.

A carrier comprising mostly PROSOLV®ODT G2 by weight may also include other separately added fillers, binders, lubricants, disintegrants, and/or glidants as necessary to optimize tablet processing and tablet properties.

### Other Excipients

In various embodiments, a carrier of a sublingual composition for the treatment of ED further comprises any combination of flavoring agent, masking agent, and/or sweetener, with the combination present at from about 0.1 wt. % to about 15 wt. %, based on the total weight of the sublingual composition for the treatment of ED, recognizing that these levels depend on the combination chosen and the types of ingredients used to mask certain unpleasant tastes and to make the overall experience of sublingual administration acceptable to the user. In some instances, a combination of sucralose (granular) at from about 0.1 wt. % to about 0.5 wt. % and flavoring agent such as peppermint powder at from about 2.5 wt. % to about 7.5 wt. % suffices to mask any unpleasant taste of the sublingual composition for the treatment of ED and make the product overall acceptable to users in taste panels.

In various embodiments, a sublingual composition for the treatment of ED includes a sweetener such as sucralose but no flavoring agent. In other embodiments, a sublingual composition for the treatment of ED includes a flavoring agent such as peppermint but no sweetener. In other embodiments, a sublingual composition for the treatment of ED includes both a flavoring agent such as peppermint and a sweetener such as sucralose.

In various embodiments, a carrier for a sublingual composition for the treatment of ED comprises a sweetener. Examples of a sweetener include, for example, stevia, aspartame, sucralose, acesulfame, or saccharin. The sweetness of these artificial substances may be amplified by ammonium glycyrrhizinate (MAG, MagnaSweet®), which also reduces off-tastes. In other examples, a sweetener for sublingual compositions for the treatment of ED may be a common sugar or sugar alcohol typically used in sweetening foods and medicines, such as sucrose, fructose, glucose, lactose, xylitol, mannitol, sorbitol, erythritol, and syrups therefrom, and so forth, noting that some of these substances find use in tablet compositions as fillers, and a composition may be deemed sweet tasting enough simply from the filler including a sugar. Any of the natural and artificial sweeteners, alone or in combination, are used in the sublingual composition for the treatment of ED at a total wt. % level sufficient to provide a consumer acceptable experience when using the sublingual ED composition. For a review of these concepts, see H. Sohi, “Taste Masking Technologies in Oral Pharmaceuticals: Recent Developments and Approaches,” Drug Development and Industrial Pharmacy, 30(5), 429-448 (2004).

In various embodiments, a carrier for a sublingual composition for the treatment of ED comprises any one or combination of flavoring agent, sweetener, buffer (or acidic agent and/or alkali agent), colorant, transmucosal permeation enhancer, stabilizer, preservative, or other pharmaceutically acceptable excipient. Any of these materials not specifically mentioned herein may be found in “Handbook of Pharmaceutical Excipients, 6^{th} Edition, R. C Rowe, et al., editors, Pharmaceutical Press, London, 2009.

Suitable flavoring agents can include, for example, flavors, such as, natural flavors, artificial flavors, and combinations thereof. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also include, for example, artificial, natural and synthetic fruit flavors such as vanilla, citrus oils (e.g., lemon, orange, lime, and grapefruit), and fruit essences (e.g., apple, pear, peach, grape, strawberry, raspberry, cherry, plum, pineapple, and apricot), and the like, and combinations thereof. Many oil flavorings are available absorbed onto powdered carriers for ease of use in powder formulations, or oils can be adsorbed into a mixture of powders being mixed in a blender, such as by addition through a spray bar configured within a ribbon mixer. In other examples, such as powdered peppermint, the leaves of the plant are dried and finely powdered, and that resulting powder used in pharmaceutical compositions.

Other flavoring agents and fragrant aromatics that may be included individually or in combination include, for example, anethole, menthol, menthone, menthyl acetate, eucalyptol, borneol, borneol acetate, camphor, 1,8-cineole, cinnamaldehyde, benzaldehyde, citral, thujone, eugenol, limonene, geraniol, citronellol, citronellal, pinene, linalool, thymol, carvone, caryophyllene, linalyl acetate, methyl salicylate, and mixtures thereof. Also, substances that provide scent and flavor include, for example, 3,3,5-trimethylcyclohexanol, methoxycyclohexanol, benzyl alcohol, anise alcohol, cinnamyl alcohol, β-phenyl ethyl alcohol (2-phenylethanol), cis-3-hexenol, musk xylol, isoeugenol, methyl eugenol, α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, hydroxycitronellal, benzaldehyde, methyl nonyl acetaldehyde, dodecanol, α-hexylcinnamic aldehyde, undecenal, heliotropin, vanillin, ethyl vanillin, methyl amyl ketone, methyl β-naphthyl ketone, methyl nonyl ketone, musk ketone, diacetyl, acetyl propionyl, acetyl butyryl, acetophenone, p-methyl acetophenone, ionone, methyl ionone, amyl butyrolactone, diphenyl oxide, methyl phenyl glycidate, γ-nonyl lactone, coumarin, cineole, ethyl methyl phenyl glycidate, methyl formate, isopropyl formate, linalyl formate, ethyl acetate, octyl acetate, methyl acetate, benzyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, β-phenylethyl acetate, trichloromethylphenyl carbinyl acetate, terpinyl acetate, vetiveryl acetate, and mixtures thereof. Some of these compounds are going to function as masking agents, and there is no attempt here to distinguish from what might purely be a flavoring agent and what might purely be a masking agent since a masking agent is likely to have some flavor profile.

Suitable buffers may comprise one or more acidifying agents or alkaline agents as necessary to neutralize various co-ingredients, form salts of various co-ingredients, and/or achieve a particular pH target for the composition, such as to adjust the local environment in the oral cavity. Combinations of various acidifying agents and alkaline agents may be used to create buffering systems that stabilize the desired final pH of an aqueous solution formed from a powdered composition. Buffers may be mixed buffers, meaning that the alkaline agent is not necessarily the conjugate base of the acidifying agent.

Exemplary acidifying agents for use in the present compositions include, for example, organic acids of any molecular weight and mineral acids (inorganic acids such as HCl), and mixtures thereof. Organic acids may include mono-carboxylic acids, di-carboxylic acids, or tri-carboxylic acids, and may be saturated or may have any degree of unsaturation. For example, organic acids for use in various embodiments of the composition in accordance to the present disclosure may include, for example, formic acid, carbonic acid, acetic acid, lactic acid, oxalic acid, propionic acid, valeric acid, enanthic acid, pelargonic acid, butyric acid, lauric acid, docosahexaenoic acid, eicosapentaenoic acid, pyruvic acid, acetoacetic acid, benzoic acid, salicylic acid, aldaric acid, fumaric acid, glutaconic acid, traumatic acid, muconic acid, malonic acid, malic acid, succinic acid, glutaric acid, adipic acid, pimelic acid, suberic acid, azelaic acid, abietic acid, pimaric acid, sebacic acid, phthalic acid, isophthalic acid, terephthalic acid, maleic acid, citric acid, and combinations thereof. Pharmaceutical actives herein may be in the form of a salt, such as an acetate salt or a hydrochloride salt. Such salts may be obtained separately and added into a composition, or such salts may be formed in situ.

Exemplary alkaline materials include any organic amines, NH₃, alkali metal or alkaline earth hydroxide, any conjugate bases of any organic acids (e.g. R-COO⁻), and any of the salts of carbonic acid, phosphoric acid, nitric acid and sulfuric acid, and any mixtures thereof. For example, alkaline materials for use in various embodiments of the composition may include, for example, NaOH, KOH, sodium acetate, sodium succinate, disodium succinate, monosodium citrate, disodium citrate, trisodium citrate, NaH₂PO₄, Na₂HPO₄, Na₃PO₄, KH₂PO₄, K₂HPO₄, K₃PO₄, NaHSO₄, Na₂SO₄, KHSO₄, K₂SO₄, NaHCO₃, Na₂CO₃, KHCO₃, K₂CO₃, NaH₃P₂O₇, Na₂H₂P₂O₇, Na₃HP₂O₇, NaAP₂O₇, KH₃P₂O₇, K₂H₂P₂O₇, K₃HP₂O₇, K₄P₂O₇, and mixtures thereof. Any of these chemical species may exist as various hydrates when purchased as raw materials for use in the present compositions.

Exemplary colorants include the pharmaceutically acceptable colors used for powdered and tableted pharmaceutical dosage forms, such as the United States Food & Drug Administration (FDA) certified colors for use in pharmaceutical compositions. Examples include FD&C colors and some D&C colors. These colorants, if even found necessary to include in the powdered sublingual compositions for tableting, are simply for aesthetic reasons. In various examples, sublingual tablets for the treatment of ED as disclosure herein comprise no colorants and appear white or slightly off-white from the color of the filler.

In various embodiments, sublingual composition for the treatment of ED may also include a transmucosal permeation enhancer to accelerate absorption of the ED medication(s) sublingually. Suitable transmucosal permeation enhancers include, for example, surfactants that assist bio-absorption, including, for example, fatty acids and/or esters or salts thereof, bile acids and/or salts thereof. In some instances, a permeation enhancer may be a polysorbate or sorbate ester. For a review of transmucosal permeation enhancers that find use herein, see B. Aungst, “Comparison of the effects of various transmucosal absorption promoters on buccal insulin delivery,” International Journal of Pharmaceutics, 53(3), 227-235 (1989).

Stabilizers and preservatives for oral compositions include the parabens, sorbitol, sodium benzoate, benzoic acid, sorbic acid, potassium sorbate, propionic acid, and combinations thereof. Antioxidants include, for example, Vitamin C, Vitamin E, butylated hydroxyanisole (BHA), butylated hydroxytoluene (BHT), and propylgallate. For a review see, I. Himoudy, “Preservatives and their role in pharma and clinical research,” International Journal of Pharma Sciences and Scientific Research, 2:4, 134-151 (2016).

### Synergistic Additives

In various embodiments, compositions for the treatment of ED further comprise a vitamin. In certain examples, ED medications include at least one of Vitamin B12 and Vitamin B6.

Synergistic vitamins are particularly useful in the compositions comprising a combination of the short-acting PDE5 active vardenafil and the long-acting PDE5 active tadalafil, optionally in further combination with oxytocin and apomorphine. The combination of vardenafil and tadalafil mitigates the need for continual dosing, and a full body effect is obtained by adding Vitamin B12 and/or Vitamin B6. Vitamin B6 catalyzes production of neurotransmitters for proper brain function, synergistically working with the oxytocin and apomorphine to help with mood regulation to decrease ED symptoms and depression and consequently increase sexual arousal and response. Vitamin B12 words to metabolize homocysteine into methionine, and it is known that excessive levels of homocysteine can cause ED.

In various embodiments, a composition for the treatment of ED comprises at least one PDE5 inhibitor and/or dopamine agonist in combination with at least one of Vitamin B6 and Vitamin B12. The amount of either vitamin is targeted to the recommended daily allowance (RDA) given the number of ED tablets prescribed. For example, the level of Vitamin B6 in an ED tablet can be set such that an individual consumes about 1.5 to 2 mg Vitamin B6 per day. Similarly, the level of Vitamin B12 in an ED tablet can be set such that an individual consumes about 2 to 3 μg Vitamin B12 per day.

TABLE 1 sets forth general embodiments of a sublingual composition for the treatment of ED. The compositions have the physical form of a powder, and the embodiments within these ranges are powders that are amendable to tableting in a tablet press into hard small tablets for sublingual administration.

TABLE 1
Sublingual Compositions for the Treatment of ED

| Ingredient (wt. %) | Preferred wt. % ranges | |
|---|---|---|
| PDE5 Inhibitor and/or Dopamine Agonist | 0.05 to 10 | |
| (drug active(s) in total wt. %) | | |
| Glidant | 0 to 1 | |
| Lubricant | 0 to 5 | |
| Flavorings, sweetener and/or masking agent(s) | 0 to 10 | |
| (in total wt. %) | | |
| Synergistic vitamin(s) (Vitamin B6 and/or | 0 to 1 | |
| Vitamin B12) | | |
| Filler and disintegrant mixture¹ | q.s. | |
| Total | 100 | wt. % |
| Physical Appearance | Free-flowing powder | |
| Transmucosal route of administration | Sublingual in tablet form | |
| Recommended tablet weight | 50-400 | mg |
| Preferred tablet weight | 100-275 | mg |

### Table 1 Notes:

(1) In various embodiments, this filler and disintegrant mixture may comprise a mixture of microcrystalline cellulose, colloidal silicon dioxide, mannitol, fructose, and crospovidone, available for example under the trade name PROSOLV® ODT G2 from JRS Pharma, Cedar Rapids, IA. In some instances, the microcrystalline cellulose and colloidal silicon dioxide can be replaced by silicified microcrystalline cellulose.

### Exemplary Compositions, Dosage Forms, and Methods of Administration

TABLE 2 sets forth preferred compositions for a sublingual compositions usable for the treatment of ED. The example compositions are loose powders proven amendable to tableting into 100 mg or 275 mg tablets using a standard tablet press, as detailed below.

TABLE 2
Exemplary Sublingual Compositions for ED and Dosages:

| | Compositions Amenable to Tableting into Sublingual Tablets | | | |
|---|---|---|---|---|
| Ingredients (in wt. %) | 1² | 2 | 3 | 4 |
| Oxytocin (10 UNIT/MG)³ | 0.89 | 0.89 | 0.91 | 5.05 |
| Vardenafil (actives) | 3.57 | 3.57 | -0- | -0- |
| Tadalafil (actives) | 3.57 | -0- | 4.55 | -0- |
| Apomorphine (actives) | 0.71 | 0.71 | -0- | -0- |
| Silica, untreated fumed | 0.71 | 0.71 | 0.73 | -0- |
| Sodium stearyl fumarate | 2.68 | 2.68 | 2.73 | 3.03 |
| PROSOLV ® ODT G2⁴ | 80.36 | 83.93 | 85.27 | 84.44 |
| Sucralose, granular | 0.36 | 0.36 | 0.36 | 0.40 |
| Peppermint (powdered) | 5.36 | 5.36 | 5.45 | 7.07 |
| Ammonium glycyrrhizinate (MAG) | 1.79 | 1.79 | -0- | -0- |
| Total | 100.00 | 100.00 | 100.00 | 100.00 |
| Appearance | White powder | White powder | White powder | White powder |
| Tablet after tableting in a tablet press | 275 mg white | 275 mg white | 275 mg white | 100 mg white |
| | tablets | tablets | tablets | tablets |
| Active oxytocin per tablet | about 0.042 mg | about 0.042 mg | about 0.042 mg | about 0.084 mg |
| Active vardenafil per tablet | about 10 mg | about 10 mg | -0- | -0- |
| Active tadalafil per tablet | about 10 mg | -0- | about 12.5 mg | -0- |
| Active apomorphine per tablet | about 2 mg | about 2 mg | -0- | -0- |

### Table 2 Notes:

(2) Composition 1 has the unique capability to treat not only the physiological aspect of ED but also the mental/cognitive aspects, as the composition has two PDE5 inhibitors and cognitive medications in combination. This formula is also for individuals who desire a long acting medication. Composition 1 is able to treat ED for up to 36 hours.
(3) OXYTOCIN 10 UNIT/MG/MANNITOL TRITURATION is used in the formulations disclosed. As discussed above, this ingredient contains 0.0168 wt. % active peptide. It should be noted that Tablets #1-3 each deliver about 25 IU oxytocin, whereas Table #4 delivers about 50 IU oxytocin. Given a recommended dosage range of 10-50 IU for safe and effective use of oxytocin, it is likely that in certain situations, an individual may take one or two 275 mg tablets of Tablets #1, 2, or 3, and just one tablet or ½ of one 100 mg tablet of Tablet #4.
(4) PROSOLV® ODT G2 provides a mixture of microcrystalline cellulose, colloidal silicon dioxide, mannitol, fructose, and crospovidone, wherein the combined amount of microcrystalline cellulose, colloidal silicon dioxide, mannitol and fructose is about 95 wt. % to about 98 wt. % of the PROSOLV® ODT G2 mixture and wherein the amount of crosprovidone is about 2 wt. % to about 5 wt. % of the PROSOLV® ODT G2 mixture.

In various embodiments, compositions according to TABLE 2 may also include Vitamin B6 and/or Vitamin B12.

The compositions of TABLE 1 and TABLE 2 are produced by simple mixing in accordance with the following procedure:

All ingredients are combined and mixed in a V-blender for about 45 minutes or until a homogenous free-flowing powder is obtained. For tableting, the powder is placed in the hopper of a Natoli NP-255 Production Tablet Press or a Stokes Model 511 tablet press calibrated to produce tablets weighing approximately 100 mg or 275 mg each depending on composition. The results of the tableting process are hard white tablets.

In various embodiments, a dose of a composition for ED is an individual sublingual tablet weighing about 50 mg to about 400 mg, or preferably about 100 mg to 275 mg, and delivering about 0.042 mg oxytocin, 10 mg vardenafil, 10 mg tadalafil, and 2 mg apomorphine per tablet.

In various embodiments, a dose of a composition for ED is an individual sublingual tablet weighing about 50 mg to about 400 mg, or preferably about 100 mg to 275 mg, and delivering about 0.042 mg oxytocin, 10 mg vardenafil, and 2 mg apomorphine per tablet.

In various embodiments, a dose of a composition for ED is an individual sublingual tablet weighing about 50 mg to about 400 mg, or preferably about 100 mg to 275 mg, and delivering about 0.042 mg oxytocin and 12.5 mg tadalafil per tablet.

In various embodiments, a dose of a composition for ED is an individual sublingual tablet weighing about 50 mg to about 400 mg, or preferably about 100 mg, and delivering about 0.084 mg oxytocin per tablet.

### Dosage Regimens and Dosage Amounts

In various embodiments, methods for treating ED are described, including aspects of (a) treating erectile dysfunction in an individual in need thereof, (b) inducing an erection in an individual in need thereof, and (c) sustaining an erection for a period of time sufficient for sexual intercourse in an individual in need thereof. In general, sublingual tablets according to the present disclosure are used to treat ED.

In various embodiments, an individual in need thereof has been diagnosed or otherwise evaluated by a clinical professional, typically a urologist, as having erectile dysfunction. These individuals in need thereof may seek therapeutic treatments of their ED with a regimen comprising sublingual tablets in accordance with the present disclosure. See, for example, D. Hatzichristou, et al., “Diagnostic Steps in the Evaluation of Patients with Erectile Dysfunction,” J. Urology, 168 (2), 615-620, 2002.

In various embodiments, a method for treating ED in an individual in need thereof comprises sublingually administering to the individual a therapeutically effective amount of a sublingual composition for ED. In various examples, the individual in need thereof is clinically diagnosed as having ED.

Sublingual versions of ED medications according to the present disclosure will improve the onset of the expected outcome for an individual in need thereof, as the dosage form need not go through first-pass metabolism, so the patient is able to use a lesser amount of active medication for a therapeutic effect. Compositions herein that deliver a combination of oxytocin, vardenafil, 1tadalafil, and apomorphine treat both the physiological issues as well as the mental/cognitive issues that can go along with ED.

In various embodiments, the therapeutically effective amount of the sublingual composition for ED comprises an amount sufficient to sublingually provide 2.5 mg to 20 mg vardenafil, 2.5 mg to 20 mg tadalafil, 1 mg to 3 mg apomorphine, and/or 0.0168 mg to 0.084 mg oxytocin. In various examples, the therapeutically effective amount of the sublingual composition for ED treatment comprises from one-half (½) of one tablet to up to about five (5) 100 or 275 mg sublingual tablets as needed, depending on individual needs and the active or actives combination present in the sublingual tablet.

For example, a therapeutically effective amount for an individual seeking improvement to their sexual performance may be one (1) to up to about five (5) 275 mg tablets, administered sublingually prior to engagement in sexual activity, wherein each 275 mg tablet provides one of: (a) about 0.042 mg oxytocin, about 10 mg vardenafil, about 10 mg tadalafil, and about 2 mg apomorphine in combination; (b) about 0.042 mg oxytocin, about 10 mg vardenafil, and about 2 mg apomorphine in combination; or (c) about 0.042 mg oxytocin and about 12.5 mg tadalafil in combination. In additional examples, a therapeutically effective amount for an individual seeking improvement to their sexual performance may be one-half (½) of one tablet up to about one (1) 100 mg tablet, administered sublingually prior to engagement in sexual activity, wherein each 100 mg tablet provides about 0.84 mg oxytocin as the only pharmaceutical active in the tablet.

In various embodiments, a method of promoting and sustaining an erection in an individual in need thereof comprises sublingually administering to the individual a therapeutically effective amount of a sublingual composition for ED comprising at least one pharmaceutical active selected from the group consisting of oxytocin, vardenafil, tadalafil, apomorphine, or mixtures thereof. In various examples, the individual in need thereof is clinically diagnosed as having ED. In various embodiments, the therapeutically effective amount of the sublingual composition for the treatment of ED comprises an amount sufficient to sublingually provide: (a) from about 0.0168 mg to about 0.084 mg oxytocin, from about 2.5 mg to about 20 mg vardenafil, from about 2.5 mg to about 20 mg tadalafil, and from about 1 mg to about 3 mg apomorphine in combination; (b) from about 0.0168 mg to about 0.084 mg oxytocin, from about 2.5 mg to about 20 mg vardenafil, and from about 1 mg to about 3 mg apomorphine in combination; (c) about from about 0.0168 mg to about 0.084 mg oxytocin and from about 2.5 mg to about 20 mg tadalafil in combination; or (d) from about 0.0168 mg to about 0.084 mg oxytocin. The individual in need thereof can adjust these dosages based on the experience from prior dosing. Stated more simply, an individual can reduce or increase the number of sublingual tablets for ED depending on whether the previous experience over-delivered or under-delivered with regards to the individual's expectations.

A dose of a sublingual composition herein for treatment of ED is from about 50 mg to about 400 mg, preferably about 100 to 275 mg of a composition, with a single dose preferably comprising a single sublingually administered tablet.

Sublingual compositions useful in the treatment of ED and methods of use thereof are provided. In the detailed description herein, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for a composition or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises,” “comprising,” or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1. A sublingual composition for erectile dysfunction (ED) comprising:
from about 0.01 wt. % to about 0.10 wt. % of oxytocin actives, optionally in combination with at least one other active selected from the group consisting of vardenafil, tadalafil, apomorphine, and mixtures thereof; and
a carrier;
wherein the weight percentages are based on the total weight of the sublingual composition for erectile dysfunction (ED), and
wherein the sublingual composition is in the physical form of a loose powder amendable to tableting by compression in a tablet press.

2. The sublingual composition for erectile dysfunction (ED) of claim 1, comprising from about 0.05 wt. % to about 0.10 wt. % oxytocin in the absence of said optional actives in combination, based on the total weight of the sublingual composition for erectile dysfunction (ED).

3. The sublingual composition for erectile dysfunction (ED) of claim 1, comprising from about 2.0 wt. % to about 5.0 wt. % tadalafil and from about 0.01 wt. % to about 0.02 wt. % oxytocin in combination, based on the total weight of the sublingual composition for erectile dysfunction (ED).

4. The sublingual composition for erectile dysfunction (ED) of claim 1, comprising from about 2.0 wt. % to about 5.0 wt. % vardenafil, from about 0.01 wt. % to about 0.02 wt. % oxytocin, and from about 0.5 to about 1.0 wt. % apomorphine in combination, based on the total weight of the sublingual composition for erectile dysfunction (ED).

5. The sublingual composition for erectile dysfunction (ED) of claim 1, comprising from about 2.0 wt. % to about 5.0 wt. % tadalafil, from about 2.0 wt. % to about 5.0 wt. % vardenafil, from about 0.01 wt. % to about 0.02 wt. % oxytocin, and from about 0.5 to about 1.0 wt. % apomorphine in combination, based on the total weight of the sublingual composition for erectile dysfunction (ED).

6. The sublingual composition for erectile dysfunction (ED) of claim 1, wherein the carrier comprises a filler, a binder, a lubricant, a disintegrant, a glidant, or mixtures thereof.

7. The sublingual composition for erectile dysfunction (ED) of claim 1, wherein the carrier comprises a mixture of untreated fumed silica, sodium stearyl fumarate, microcrystalline cellulose, colloidal silicon dioxide, mannitol, fructose, and crospovidone.

8. The sublingual composition for erectile dysfunction (ED) of claim 1, wherein the carrier comprises from about 0.1 wt. % to about 5 wt. % of sodium stearyl fumarate, based on the total weight of the sublingual composition for erectile dysfunction (ED).

9. The sublingual composition for erectile dysfunction (ED) of claim 1, wherein the carrier comprises from about 0.5 wt. % to about 1 wt. % untreated fumed silica, based on the total weight of the sublingual composition for erectile dysfunction (ED).

10. The sublingual composition for erectile dysfunction (ED) of claim 1, wherein the carrier comprises from about 0.1 wt. % to about 5 wt. % crospovidone, based on the total weight of the sublingual composition for erectile dysfunction (ED).

11. A sublingual tablet for transmucosal administration comprising:
from about 2.0 wt. % to about 5.0 wt. % tadalafil;
from about 2.0 wt. % to about 5.0 wt. % vardenafil;
from about 0.01 wt. % to about 0.02 wt. % oxytocin;
from about 0.5 to about 1.0 wt. % apomorphine; and
at least about 85 wt. % of a carrier;
wherein the weight percentages are based on the total weight of the sublingual tablet.

12. The sublingual tablet of claim 11, wherein the carrier comprises a mixture of untreated fumed silica, sodium stearyl fumarate, microcrystalline cellulose, colloidal silicon dioxide, mannitol, fructose and crospovidone.

13. The sublingual tablet of claim 12, wherein each tablet weighs approximately 275 mg and wherein each tablet delivers about 0.042 mg oxytocin, 10 mg vardenafil, 10 mg tadalafil, and 2 mg apomorphine.

14. The sublingual tablet of claim 12, wherein said carrier further comprises at least one of a sweetener and flavoring agent.

15. A method for treating erectile dysfunction (ED) in an individual in need thereof, the method comprising sublingually administering to the individual a therapeutically effective amount of a sublingual composition comprising:
from about 2.0 wt. % to about 5.0 wt. % tadalafil;
from about 2.0 wt. % to about 5.0 wt. % vardenafil;
from about 0.01 wt. % to about 0.02 wt. % oxytocin;
from about 0.5 to about 1.0 wt. % apomorphine; and
remainder, a carrier;
wherein the weight percentages are based on the total weight of the sublingual composition.

16. The method of claim 15, wherein the therapeutically effective amount is an amount sufficient of the sublingual composition to deliver 2.5 mg to 20 mg vardenafil, 2.5 mg to 20 mg tadalafil, 1 mg to 3 mg apomorphine, and 0.0168 mg to 0.084 mg oxytocin.

17. The method of claim 15, wherein the sublingual composition is in the physical form of individual sublingual tablets weighing approximately 275 mg each, and wherein the therapeutically effective amount of the sublingual composition comprises from about one (1) to about five (5) sublingual tablets, taken as needed.

18. The method of claim 15, wherein the individual in need thereof is clinically evaluated to have erectile dysfunction (ED).

19. A sublingual tablet consisting essentially of:
2.5 mg oxytocin;
10 mg vardenafil;
2 mg apomorphine; and
260.5 mg of a mixture of fumed untreated silica, sodium stearyl fumarate, microcrystalline cellulose, colloidal silicon dioxide, mannitol, fructose, crospovidone, optional sweetener, and optional flavoring agent.

20. A sublingual tablet consisting essentially of:
2.5 mg oxytocin;
12.5 mg tadalafil; and
260.0 mg of a mixture of fumed untreated silica, sodium stearyl fumarate, microcrystalline cellulose, colloidal silicon dioxide, mannitol, fructose, crospovidone, optional sweetener, and optional flavoring agent.
